(19) United States
(12) Patent Application Publication
(10) Pub. No.: US 2009/0180965 A1
(43) Pub. Date: Jul. 16, 2009

(54) METHODS FOR REPAIR AND REGENERATION OF BONE MARROW

(75) Inventors: Toby M. Freyman, Waltham, MA (US); Wendy Naimark, Boston, MA (US); Maria Palasis, Wellesley, MA (US)

Correspondence Address: FULBRIGHT & JAWORSKI L.L.P., 600 CONGRESS AVE., SUITE 2400, AUSTIN, TX, 78701, US

(73) Assignee: BOSTON SCIENTIFIC SCIMED, INC., Maple Grove, MN (US)

(21) Appl. No.: 12/348,207

(22) Filed: Jan. 2, 2009

Related U.S. Application Data
(60) Provisional application No. 61/019,040, filed on Jan. 4, 2008.

(51) Int. Cl. A61K 49/00; A61K 35/12

(52) U.S. Cl. 424/9.3; 424/520; 424/9.1; 424/9.6; 424/548; 424/570; 424/569; 424/549; 424/556; 424/551; 424/553

(57) ABSTRACT

The invention is directed to a method for treating a tissue or organ in a subject by directly administering an effective amount of an exogenous, decellularized extracellular matrix or a mixture of extracellular matrix and mesenchymal stem cells into the intended site of activity, such as bone marrow cavity. In one embodiment, the invention provides methods of treating bone marrow to increase the number of circulating progenitor and stem cells. In some other embodiments of the invention, the decellularized extracellular matrix to be directly administered is configured to be a time released therapeutic.

This application claims the benefit of priority to U.S. Provisional Patent Application Ser. No. 61/019,040, filed Jan. 4, 2008, which is hereby incorporated by reference in its entirety.

### 1. FIELD OF THE INVENTION

The present invention relates generally to tissue repair and organ regeneration. In particular, the invention relates to treating defective, diseased, damaged or ischemic tissues or organs in a subject by injecting or implanting decellularized extracellular matrix of conditioned body tissue directly into the site where treatment or therapy is desired.

### 2. BACKGROUND OF THE INVENTION

Circulating progenitor and stem cells participate in both normal homeostatic functions (e.g. maintaining endothelium) and facilitate wound repair. These cells are produced in and reside in the bone marrow. The ability of bone marrow to produce stem cells declines with age. This is evidenced by the fact that stem cell numbers drop and red marrow converts to fatty, yellow marrow with increasing age.

There are many health conditions which result in a deficiency of circulating progenitor and stem cells. For example, low numbers and/or defective response by these cells has been implicated in diseases such as atherosclerosis (Werner et al., 2006, J. Cell. Mol. Med. 10:318-332) and poor repair after injury, such as with myocardial infarction (de Boer et al., 2006, Arterioscler. Thromb. Vasc. Biol. 26:1653-1659; Water et al., 2006 Circulation 109:1615-1622). Although bone marrow can be stimulated to release progenitor cells through administration of G-CSF or GM-CSF, it has been documented that in some patients, particularly those with coronary heart disease, these cells cannot be mobilized in significant numbers or are deficient once mobilized.

Therefore, there is a continuing need for agents which are safe to administer, promote regeneration of tissues, and stimulate the development of various progenitor and stem cells.

### 3. SUMMARY OF THE INVENTION

The present invention is based on the discovery that directly injecting or implanting bone marrow extracellular matrix (ECM) into a tissue or organ at the site where activity is intended guides tissue repair and regeneration in injured tissue. Injection of bone marrow extracellular matrix into a bone marrow cavity, for example, promotes bone marrow regeneration and stem cell growth. Although bone marrow stem cell reconstitution has been developed to specifically facilitate cancer therapies, such approaches only focus on treating bone marrow which has been knowingly damaged by the chemo and/or radiation therapies. These therapies also rely on the patient having otherwise healthy bone marrow or an available match donor. The present invention, however, is directed to the treatment of bone marrow dysfunction resulting from environmental factors, a disease process, or aging. Using the methods of the present invention for the treatment of bone marrow prior to radiation or chemotherapy helps to facilitate marrow recovery and/or survival. The methods of the present invention can be used along with local, controlled-release drug delivery.

In one embodiment, the present invention provides a method for treating a tissue or organ in a subject, comprising directly administering an effective amount of an exogenous, decellularized extracellular matrix (ECM) into the site of the tissue or organ of the subject. Alternatively, the tissue or organ may be examined by in vivo imaging prior to the administration of the ECM. In vivo imaging techniques include, but are not limited to, computer assisted tomography (CAT), magnetic resonance spectroscopy (MRS), magnetic resonance imaging (MRI), positron emission tomography (PET), single-photon emission computed tomography (SPECT), bioluminescence image (BLI) or equivalent.

In another embodiment, the tissue or organ is selected from the group consisting of bone marrow, spleen, and thymus.

In another embodiment, the tissue or organ is a dysfunctional tissue or organ.

In still another embodiment, the present invention provides a method for treating a tissue or organ in a subject, comprising directly administering an effective amount of ECM in combination with mesenchymal stem cells into the site of a tissue or organ of the subject. The tissue or organ may also be examined by in vivo imaging prior to the administration of the ECM in combination with the mesenchymal stem cells.

In still another embodiment, the exogenous, decellularized extracellular matrix and the mesenchymal stem cells are mixed ex vivo prior to direct administration.

In still another embodiment, the invention provides a method for treating and/or preventing diseases involving insufficiency of mature or regenerating cells in tissues, comprising administering to a patient in need thereof exogenous, decellularized bone marrow extracellular matrix into a bone marrow cavity.

In still another embodiment, the invention provides a method for treating and/or preventing diseases involving myocardial regeneration, comprising administering to a patient in need thereof exogenous, decellularized bone marrow extracellular matrix into a bone marrow cavity.

In still another embodiment, the invention provides a method of regenerating the endothelium, comprising administering to a patient in need thereof exogenous, decellularized bone marrow extracellular matrix into a bone marrow cavity.

In still another embodiment, the invention provides a method of preventing and/or treating atherosclerosis, comprising administering to a patient in need thereof extracellular matrix into a bone marrow cavity.

In still another embodiment, the site of direct administration is a bone marrow cavity. For example, the exogenous, decellularized extracellular matrix may be injected into the bone marrow cavity such as the cavity of the trabecular bone. The exogenous, decellularized extracellular matrix may also be implanted into the cavity.

In certain embodiments, the exogenous, decellularized extracellular matrix is obtained from conditioned body tissue of an animal. The body tissue is conditioned in vivo or in situ before being harvested. In certain other embodiments, the body tissue is conditioned in vitro after being harvested. If the body tissue is conditioned in vivo or in situ, conditioning may be performed locally or systemically. If the body tissue is conditioned in vitro, conditioning may be performed in a bioreactor. The conditioned body tissue is given a period of time before and/or after harvest to produce the biological material in an amount of interest. The amount of biological material produced by the body tissue may be monitored before, during or after the conditioning step.

The body tissue may be conditioned using any one or more biological, chemical, pharmaceutical, physiological and/or mechanical treatment(s). In one embodiment, the body tissue is biologically conditioned by transfecting the body tissue with a nucleic acid. In another embodiment, the body tissue is chemically conditioned by incubating the body tissue in a hypotonic or hypertonic solution. In yet another embodiment, the body tissue is pharmaceutically conditioned by delivering a therapeutic agent to the body tissue. In yet another embodiment, the body tissue is physiologically conditioned by exposing the body tissue to heat shock. In yet another embodiment, the body tissue is mechanically conditioned by applying a force to the body tissue. In one embodiment, the force is produced by the expansion of a balloon against the body tissue.

The body tissue from a donor subject may be conditioned so that the biochemical composition and histoarchitecture of the body tissue is retained. In certain embodiments, the body tissue may be conditioned so that the biochemical composition and histoarchitecture of the body tissue from the donor subject is similar to the body tissue that is being repair, replaced and/or regenerated in a recipient subject. The body tissue may be from a mammal, such as a pig or human.

The conditioned body tissue may retain or possess new physical properties such as strength, resiliency, density, insolubility, and permeability as compared to the unconditioned body tissue. The conditioned body tissue may also contain a biological material in an amount different than the amount of the biological material that the body tissue would produce absent the conditioning. In a specific embodiment, the biological material is a growth factor, such as vascular endothelial growth factor (VEGF). In another specific embodiment, the biological material is an extracellular matrix protein, such as elastin.

The harvested and conditioned body tissue may be decellularized using a combination of physical, chemical, and biological processes. The methods of the present invention involve the steps of decellularization by removing native cells, antigens, and cellular debris from the extracellular matrix of the body tissue. In one embodiment, an enzyme treating step is involved.

The body tissue may be further processed after decellularization to facilitate administration, injection or implantation. For examples, the decellularized extracellular matrix can be dried, concentrated, diluted, lyophilized, cryopreserved, electrically charged, sterilized, etc. In one embodiment, the decellularized extracellular matrix is suspended in a saline solution as a final product.

The invention also relates to the administration, injection or implantation of the decellularized extracellular matrix of conditioned body tissue into a subject in need thereof. The decellularized extracellular matrix of the invention may be administered, injected or implanted alone or in combination with other therapeutically or prophylactically effective agents useful for treating, managing or preventing a disease or condition that requires tissue or organ repair, restoration and/or strengthening may be delivered to the body tissue before and/or after conditioning and/or harvesting.

In certain embodiments, the decellularized extracellular matrix of the invention may be administered, injected or implanted in combination with isolated, homogeneous mesenchymal stem cells into the site of the tissue or organ of the subject. The exogenous, decellularized extracellular matrix and the isolated, homogeneous mesenchymal stem cell may be mixed ex vivo prior to administration.

The decellularized extracellular matrix may also be administered, injected or implanted before, during or after treatment with other methods of repairing, regenerating and/or strengthening of the diseased, defected, damaged or ischemic tissue or organ. In particular, the decellularized extracellular matrix may be used to promote angiogenesis and/or repair, replace or regenerate cells, tissues or organs, such as but not limited to lymph vessels, blood vessels, heart valves, myocardium, pericardium, pericardial sac, dura mater, meniscus, omentum, mesentery, conjunctiva, umbilical cords, bone marrow, bone pieces, ligaments, tendon, tooth implants, dermis, skin, muscle, nerves, spinal cord, pancreas, gut, intestines, peritoneum, submucosa, stomach, liver, and bladder.

The decellularized extracellular matrix of the present invention can also be used to form a tissue regeneration scaffold for implantation into a subject. The tissue regeneration scaffold may be used as a therapeutic to treat diseases or conditions that may benefit from improved angiogenesis, cell proliferation and/or tissue regeneration and/or strengthening. Such diseases or conditions include but are not limited to, burns, ulcer, trauma, wound, bone fracture, diabetes, psoriasis, arthritis, asthma, cystitis, inflammation, infection, ischemia, restenosis, stricture, atherosclerosis, occlusion, stroke, infarct, aneurysm, abdominal aortic aneurysm, uterine fibroid, urinary incontinence, vascular disorders, hemophilia, cancer, and organ failure (e.g., heart, kidney, lung, liver, intestine, etc.).

The methods of the present invention can also be used in the treatment of anemia.

The methods of the present invention can also be used for preventative medicine treatments, such as in patients with high cholesterol or low stem cell function which could be treated in the early stages of coronary artery disease.

### 4. BRIEF DESCRIPTION OF THE DRAWINGS

FIG. 1 shows the effect of treatment on the bone marrow volume within the medullary canals of rats. The bone marrow compartment was increased in treated (shaded bar) rats tibias when compared control rats.

FIG. 2 shows that the trabecular bone content in the medullary canals of control rat tibias was much greater as compared to treated (shaded bar) rats.

### 5. DEFINITIONS

The term “biologically active material” encompasses therapeutic agents, such as drugs, and also genetic materials and biological materials. The genetic materials mean DNA or RNA, including, without limitation, of DNA/RNA encoding a useful protein stated below, intended to be inserted into a human body including viral vectors and non-viral vectors. The biological materials include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transfenin, cytotactin, cell binding domains (e.g., RGD), and tenascin. Currently used BMP's include BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells. Biologically active materials also include non-genetic therapeutic agents, such as:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, and acetylsalicylic acid. amlodipine and doxazosin;
anti-inflammatory agents such as glucocorticoids, betamethasone, dexamethasone, prednisolone, corticosterone, budesonide, estrogen, sulfasalazine, mesalamine, and statins;
antineoplastic/antiproliferative/anti-mitotic agents such as paclitaxel, 5-fluorouracil, cisplatin, vinblastine, cladribine, vincristine, epothilones, methotrexate, azathioprine, adriamycin and mutamycin; endostatin, angiostatin and thymidine kinase inhibitors, taxol and its analogs or derivatives;
anesthetic agents such as lidocaine, bupivacaine, and ropivacaine;
anti-coagulants such as D-Phe-Pro-Arg chloromethyl keton, an RGD peptide-containing compound, heparin, antithrombin compounds, platelet receptor antagonists, antithrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents; vasodilating agents; and agents which interfere with endogenous vasoactive mechanisms;
anti-oxidants, such as probucol;
antibiotic agents, such as penicillin, cefoxitin, oxacillin, tobranycin, rapamycin;
angiogenic substances, such as acidic and basic fibroblast growth factors, estrogen including estradiol (E2), estriol (E3) and 17-Beta Estradiol; and
drugs for heart failure, such as digoxin, beta-blockers, angiotensin-converting enzyme (ACE) inhibitors including captopril, enalopril, and statins and related compounds.

The term “decellularized extracellular matrix” and the term “acellular extracellular matrix” are used interchangeably throughout the specification.

As used herein, the term “therapeutically effective amount” refers to that amount of the therapeutic agent sufficient to treat or manage defective, diseased, damaged or ischemic tissues or organs. A therapeutically effective amount may refer to the amount of therapeutic agent sufficient to delay or minimize the onset of symptoms associated with defective, diseased, damaged or ischemic tissues or organs. A therapeutically effective amount may also refer to the amount of the therapeutic agent that provides a therapeutic benefit in the treatment or management of the defective, diseased, damaged or ischemic tissues or organs. Further, a therapeutically effective amount with respect to a therapeutic agent of the invention means that amount of therapeutic agent alone, or in combination with other agents or therapies, that provides a therapeutic benefit in the treatment or management of defective, diseased, damaged or ischemic tissues or organs. Used in connection with an amount of the decellularized extracellular matrix of the invention, the term can encompass an amount that improves overall therapy, reduces or avoids unwanted effects, or enhances the therapeutic efficacy of or synergies with another therapeutic agent.

As used herein, the term “prophylactically effective amount” refers to that amount of the prophylactic agent sufficient to result in the prevention of the occurrence of defective, diseased, damaged or ischemic tissues or organs. A prophylactically effective amount may also refer to the amount of prophylactic agent sufficient to prevent the occurrence or recurrence of defective, diseased, damaged or ischemic tissues or organs in a patient, including but not limited to those (genetically) predisposed. A prophylactically effective amount may also refer to the amount of the prophylactic agent that provides a prophylactic benefit in the prevention of defective, diseased, damaged or ischemic tissues or organs. Further, a prophylactically effective amount with respect to a prophylactic agent of the invention means that amount of prophylactic agent alone, or in combination with other agents or therapies, that provides a prophylactic benefit in the prevention of the occurrence or recurrence of defective, diseased, damaged or ischemic tissues or organs. Used in connection with an amount of the decellularized extracellular matrix of the invention, the term can encompass an amount that improves overall prophylaxis or enhances the prophylactic efficacy of or synergies with another prophylactic agent.

As used herein, the terms “subject” and “patient” are used interchangeably. As used herein, a subject is a mammal such as a non-primate (e.g., cows, pigs, horses, cats, dogs, rats, etc.) and a primate (e.g., monkey and human), particularly a human.

As used herein, the term “body tissue” broadly encompasses any or a number of cells, tissues or organs.

As used herein, the term “repair” relates to the restoration of defective, diseased, damaged or ischemic tissues or organs to a sound or healthy stage by replacing a part or putting together what is defective, diseased, damaged or ischemic by synthesizing and incorporating additional normal cells, tissue or organ components to increase the size and/or strength of the defective, diseased, damaged or ischemic tissue or organ.

As used herein, the term “replace” relates to the substitution of defective, diseased, damaged or ischemic tissues or organs with newly synthesized cells, tissue or organ components facilitated by the decellularized extracellular matrix of the present invention.

As used herein, the term “regenerate” relates to the regrowth and/or reconstitution of defective, diseased, damaged or ischemic tissues or organs.

As used herein, the term “strengthen” relates to the making stronger of the defective, diseased, damaged or ischemic tissues or organs.

As used herein, the terms “biological material” and “biologically active material” are used interchangeably. Examples of a biological material include, but are not limited to, vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor of matrix metalloproteinase (TIMP), cytokines, interleukins, lymphokines, interferon, integrin, collagen (all types), elastin, fibrillins, fibronectin, laminin, glycosaminoglycans, vitronectin, proteoglycans, transferrin, cytotactin, cell binding domains (e.g., RGD), and tenascin.

As used herein, the term “analog” refers to a polypeptide that possesses a similar or identical function as a particular protein (e.g., vascular endothelial growth factor), or a fragment thereof, but does not necessarily comprise a similar or identical amino acid sequence or structure of that protein or a fragment thereof. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a protein or a fragment thereof as described herein; (b) a polypeptide encoded by a nucleotide sequence that hybridizes under stringent conditions to a nucleotide sequence encoding a protein or a fragment thereof as described herein of at least 20 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a protein or a fragment thereof as described herein. A polypeptide with similar structure to a protein or a fragment thereof as described herein refers to a polypeptide that has a similar secondary, tertiary or quaternary structure of a protein or a fragment thereof as described herein. The structure of a polypeptide can be determined by methods known to those skilled in the art, including but not limited to, X-ray crystallography, nuclear magnetic resonance, and crystallographic electron microscopy.

As used herein, the term “derivative” refers to a polypeptide that comprises an amino acid sequence of a protein, such as vascular endothelial growth factor, a fragment of the protein, an antibody that immunospecifically binds to the protein, or an antibody fragment that immunospecifically binds to the protein which has been altered by the introduction of amino acid residue substitutions, deletions or additions. The term “derivative” as used herein also refers to the protein, a fragment of the protein, an antibody that immunospecifically binds to the protein, or an antibody fragment that immunospecifically binds to the protein which has been modified, i.e., by the covalent attachment of any type of molecule to the polypeptide. For example, but not by way of limitation, by glycosylation, acetylation, pegylation, phosphorylation, amidation, derivatization by known protecting/blocking groups, proteolytic cleavage, linkage to a cellular ligand or other protein, etc. A derivative may also be modified by chemical modifications using techniques known to those of skill in the art, including, but not limited to specific chemical cleavage, acetylation, formylation, metabolic synthesis of tunicamycin, etc. Further, the derivative may contain one or more non-classical amino acids. In one embodiment, the derivative possesses a similar or identical function as the protein of interest. In another embodiment, the derivative has an altered activity when compared to an unaltered protein. For example, a derivative antibody or fragment thereof can bind to its epitope more tightly or be more resistant to proteolysis.

As used herein, the term “fragment” refers to a peptide or polypeptide comprising an amino acid sequence of at least 20 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a protein, such as vascular endothelial growth factor.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. The NBLAST and XBLAST programs of Altschul et al., 1990, J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used.

Another example of a mathematical algorithm utilized for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti, 1994, Comput. Appl. Biosci. 10:3-5; and FASTA described in Pearson and Lipman, 1988, Proc. Natl. Acad. Sci. 85:2444-8. Within FASTA, ktup is a control option that sets the sensitivity and speed of the search.

### 6. DETAILED DESCRIPTION OF THE INVENTION

The present invention provides new methods for treating tissues or organs such as bone marrow as to facilitate the repair and regeneration of the tissues or organs. In particular, the present invention provides methods of direct administration of exogenous and decellularized extracellular matrix into the bone marrow cavity of bone marrow to restore normal production of circulating stem and progenitor cells. The spleen and thymus are also known to support stem cell proliferation and can be an alternate target for the methods and therapies of the present invention.

It is well known that circulating stem and progenitor cells play important roles in the maintenance of normal homeostatic functions. The methods described and claimed herein therefore are especially useful for patients suffering various disease associated with bone marrow and low number of circulating stem and progenitor cells. These patients include, but are not limited to, anemic patients, patients needing vascular regeneration after devastating burn injuries, patients presenting with high cholesterol or low stem cell function who may be treated in early stage of coronary artery disease, patients undergoing radiation or chemotherapy who need bone marrow derived stem and progenitor cells to facilitate marrow recovery or survival, patients who need progenitor cells for the prevention of atherosclerotic diseases, patients with ischemic cardiomyopathy who may need bone marrow derived mononuclear cells to improve cardiovascularization and/or cardiac regeneration, and the like.

The present invention relates to the use of decellularized extracellular matrix of body tissue, including conditioned body tissue. In certain embodiments, the body tissue of a donor subject is conditioned in vivo or in situ before harvest. In certain embodiments, the body tissue of a donor subject is first harvested and then conditioned in vitro, such as in a bioreactor. The conditioned body tissue is given a period of time to produce a in an amount different than the amount that is produced by a body tissue absent the conditioning. The conditioned body tissue may be decellularized by at least one or a combination of physical, chemical and/or biological step(s). In one embodiment, the decellularized conditioned body tissue is rid of cellular components and only retains the extracellular matrix and the biological material of interest. In certain embodiments, the decellularized conditioned body tissue can be further processed prior to its use.

The decellularized extracellular matrix may be grafted directly onto the site of a defective, diseased, damaged or ischemic tissue or organ. The decellularized extracellular matrix may also be processed into a formulation and injected at a site in need of treatment. The decellularized extracellular matrix may further be used in a tissue regeneration scaffold for implantation into a subject. In addition, the decellularized extracellular matrix can be part of a medical device, such as a stent, an artificial heart, a ventricular restraining device, or an aneurysm coil for implantation into a subject. For instance, the decellularized extracellular matrix can be coated onto the medical device, such as by spray coating or dip coating, or incorporated into a component of the medical device. For a more complete description of using ECM with implantable medical devices, see U.S. Application Publication No. US2005/0181016, published on Aug. 18, 2005, which is herein incorporated by reference in its entirety.

Although not to be limited in theory, the decellularized extracellular matrix provides a microenvironment and contains important biological materials that promote the efficient and effective repair, regeneration and/or strengthening of cells, tissues or organs.

For clarity of disclosure, and not by way of limitation, the detailed description of the invention is divided into the subsections which follow.

6.1 Direct Administration Of ECM

The present invention provides methods for enhancing the repair and regeneration of tissues or organs through direct delivery of exogenous, decellularized extracellular matrix (ECM) into the site of the tissue or organ where activity is intended. The methods for enhancing regeneration of tissues and organs comprise directly administering an effective amount of an exogenous, decellularized extracellular matrix into the site of the tissue or organ of the subject.

ECM is a complex structural entity surrounding and supporting cells. The extracellular matrix is found within mammalian tissues and is made up of three major classes of biomolecules: structural proteins (e.g., collagen and elastin), specialized proteins (e.g., fibrillin, fibronectin, and laminin), and proteoglycans (e.g., glycosaminoglycans). In addition to providing physical support to cells, the extracellular matrix affects cell function through mechanical and chemical signals.

Although isolated ECMs are not normally readily available, the inventors of the present invention have discovered a process for isolating and purifying ECMs in vivo and in vitro. This discovery is the subject of co-pending U.S. patent application Publication Nos. 2005/0013870, 2005/0013872, and 2005/0181016, which are all incorporated by reference herein. The methods and therapies of the invention utilize such isolated and rich source of matrix proteins and growth factors.

The methods according to the invention are fully applicable to direct administration of ECM into the intended site of activity in the tissues or organs including, but not limited to, composite tissue such as, but not limited to human hand, human finger, human larynx, joints such as knee, hip, limbs, lower extremities, and the like; solid organs and glands such as, but not limited to, heart, lung, kidney, liver, spleen, pancreas, thyroid, and the like; skin; cartilage, such as an ear; hematopoietic tissue; lymphoid tissue; bone marrow, tendons; ligaments; muscles; nerve tissue vascular tissue such as vessels; and the like, without limitation. In one embodiment, the site of direct delivery of ECM includes bone marrow cavity, spleen and thymus. The ECM of the above methods may be delivered by any acceptable means that results in systemic delivery. However, delivery of the ECM is best accomplished by intra-tissue or organ delivery. This is because cells, such as bone marrow-derived stem cells, progenitor cells or mesenchymal cells, for example hematopoietic stem or progenitor cells, or mesenchymal cells, do not stay compartmentalized, but tend to migrate and/or proliferate to the adjacent recipient bone. Intra-tissue or organ delivery thus provides a means of attracting those cells to the site where ECM, which may serve as a bone scaffold, is directly administrated.

The intra-tissue or organ delivery of ECM can be performed, for example, by direct injection, such as by a large gage (18 gage or greater) syringe, into the bone marrow of the recipient. This can be accomplished by the use of minimally invasive techniques involving drilling a small hole into the patient's bone, flushing out a small volume of recipient dysfunctional or fatty bone marrow to create the “space” for the recipient bone marrow, and injecting the ECM into the small cavity created.

Several techniques may be employed to remove fatty marrow prior to delivery of ECM. For example, a liposuction-like technique can be used to provide space for the new marrow. Specifically, access to the marrow cavity is achieved using a needle (or other established methods) and vacuum is used to aspirate the fatty or dysfunctional marrow. Pre-treatment with ultrasound to facilitate breakup and removal is also contemplated. Removal of fatty marrow may also be accomplished chemically (e.g. ionic or non-ionic detergents, alcohol). These chemicals can be administered, retrieved and the cavity rinsed to remove residual chemical. This process may be used in addition to or in combination with liposuction or ultrasound energy. Fatty marrow may also be removed via radiation therapy. In addition, inclusion of a carrier matrix, gel or scaffold in the injectate or implant may also be employed. Examples of such carriers include self assembling matrices (thermal set, two-component chemical set (fibrin-thrombin, alginate), in situ reaction (ion induced self assembling peptides (RADA)). Additionally, a cell seeded scaffold (collagen, PLGA, etc.), protein loaded scaffold may be implanted into the deficient marrow space.

Alternatively, a pumping system may be used for the intra-tissue or organ transfer of ECM. Any pumping system that is medically and pharmaceutically acceptable, and which can transfer tissue or cell from donor to recipient, may be used. For example, a pressurized pumping system may be used wherein a subject's dysfunctional bone marrow is pumped out and ECM is pumped into the subject's bone.

The ECM may be delivered in crude form, i.e. they may comprise other biological materials. The biological materials include, but are not limited to, vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor of matrix metalloproteinase (TIMP), interferon, interleukins, cytokines, integrin, collagen (all types), elastin, fibrillins, fibronectin, laminin, glycosaminoglycans, vitronectin, proteoglycans, transferrin, cytotactin, cell binding domains, tenascin, and lymphokines, activated platelets (which release factors that initiate the healing process), fibrin (a key component of the provisional matrix present in newly formed and healing wounds), or the like.

The ECM composition may also include small molecules such as statins or hedgehog agonists, which facilitate stem cell proliferation and function. In one embodiment of the present invention, these small molecules are formulated to facilitate their controlled, local release into the bone marrow, e.g., drug-loaded microspheres, matrix-bound drug, coated microspheres or other implants. The ECM composition may also contain therapeutic proteins that can be control-released locally to the bone marrow area.

In one embodiment of the invention, a method is provided for exogenous, acellular ECM to be directly injected into the bone marrow cavity of the patient, such as the long bones, sternum, ribs and/or vertebrae. More specifically, a stiff needle may be used to penetrate the cortical bone and gain access to the marrow space (trabecular bone). This method allows preservation of the microenvironment for the ECM during treatment.

The direct administration of ECM in the crude form into the bone marrow cavity such as the trabecular bone allows not only for the engraftment of the EMC, but also provides a natural matrix as an ideal microenvironment for cell engraftment for subsequent cell repopulation and trafficking. This method of intra-bone marrow cavity delivery thus is simple, minimally invasive, and can be easily implemented into clinical practice in humans.

The amount of ECM to be delivered to the patient will depend on numerous factors such as, but not limited to, the type and condition of the recipient, the route of administration, and the like as described above, and can be tailored to each patient according to normal medical practice. A therapeutically effective amount of ECM injected or implanted into the recipient is that sufficient to induce bone marrow regeneration in the patient.

In the embodiments of the invention, the ECM can be derived from a young animal or human donor. A more detailed discussion of the source and preparation of the ECM is presented in the following sections of the present application. Thus, the donor and the recipient can be of the same species. In other embodiments, donor and recipient are mammals, birds, reptiles, amphibians or marsupials. In a specific embodiment, said mammals are domestic mammals. In a more specific embodiment, said domestic mammal is a canine or a feline. In another more specific embodiment, said domestic mammal is an equine, bovine, porcine species. In yet further embodiments, the recipient is a primate. In one embodiment, the recipient is a human.

6.2 Direct Administration of ECM in combination with Mesenchymal Stem Cells

The present invention further provides methods for treating a tissue or organ in a subject, comprising directly administering an effective amount of ECM in combination with mesenchymal stem cells into the site of the tissue or organ of the subject.

Direct administration of ECM in combination with mesenchymal stem cells into the tissues or organs at the site for intended activity can be accomplished in the same manner as described above. For example, the intra-tissue or organ delivery can be performed by direct injection, such as by a large gauge (18 gauge or greater) syringe, into the bone marrow of the recipient. In one embodiment of the invention, ECM and mesenchymal stem cells are directly injected into the long bones, sternum, ribs and/or vertebrae of the patient. Alternatively, a stiff needle may be used to penetrate the cortical bone and gain access to the marrow space (trabecular bone).

Although the invention is not limited thereby, mesenchymal stem cells can be isolated, purified, and expanded in culture, i.e. in vitro, to obtain sufficient numbers of cells for use in the methods described herein. See, Caplan and Haynesworth, U.S. Pat. No. 5,486,359; U.S. Pat. No. 5,197,985; U.S. Pat. No. 5,226,914; WO92/22584.

Thus in one embodiment, the human mesenchymal stem cells are obtained from bone marrow taken from an individual allogeneic to the recipient. In one embodiment, the mesenchymal cell preparation is substantially pure, i.e. is at least 95% free of allogeneic cells other than mesenchymal stem cells.

The subject human mesenchymal stem cells are obtained from the bone marrow or other mesenchymal stem cell source. Bone marrow cells may be obtained from iliac crest, femora, tibiae, spine, rib or other medullary spaces. Other sources of human mesenchymal stem cells include embryonic yolk sac, placenta, umbilical cord, fetal and adolescent skin, and blood.

Isolated and purified allogeneic human mesenchymal stem cells can be grown in an undifferentiated state through mitotic expansion in a specific medium. These cells can then be harvested and activated to differentiate into bone, cartilage, and various other types of connective tissue by a number of factors, including mechanical, cellular, and biochemical stimuli. Human mesenchymal stem cells possess the potential to differentiate into cells such as osteoblasts and chondrocytes, which produce a wide variety of mesenchymal tissue cells, as well as tendon, ligament and dermis, and this potential is retained after isolation and for several population expansions in culture. Thus, by being able to isolate, purify, greatly multiply, and then activate mesenchymal stem cells to differentiate into the specific types of mesenchymal cells desired, such as skeletal and connective tissues such as bone, cartilage, tendon, ligament, muscle, adipose and marrow stroma, see U.S. Pat. No. 5,197,985, a highly effective process exists for treating skeletal and other connective tissue disorders.

Although in one embodiment the mesenchymal stem cells are culturally expanded prior to use, it is also possible to use such mesenchymal stem cells without culture expansion. For example, mesenchymal stem cells may be derived from bone marrow and used after separation of blood cells there from, without expansion. Thus, for example, allogeneic bone marrow may be enriched in allogeneic human mesenchymal stem cells by removal of blood cells, and introduced into a patient in need thereof, e.g., for skeletal repair.

Alternatively, the mesenchymal stem cells may be purified and enriched, such as by cell sorting techniques that are well known in the art, prior to intra-tissue or organ delivery. Transplantation of vascularized bone containing bone marrow may also be used to effect transplantation of bone marrow, and stem cells contained therein, for example, mesenchymal cells, B cells, chimeric cells derived from the recipient, etc.

The cells can be suspended in an appropriate diluent, at a concentration of from about 0.5 to about 5×10⁶ cells/ml. Suitable excipients for such solutions are those that are biologically and physiologically compatible with the recipient, such as buffered saline solution. Other excipients include water, isotonic common salt solutions, alcohols, polyols, glycerine and vegetable oils. The composition for administration must be formulated, produced and stored according to standard methods complying with proper sterility and stability.

The dose of the ECM and mesenchymal stem cells varies within wide limits and will, of course, be fitted to the individual requirements in each particular case. The amount of ECM and the number of cells used will depend on the weight and condition of the recipient and other variables known to those of skill in the art. In most cases, ECM and the allogeneic mesenchymal stem cells are delivered to the site of desired treatment or therapy and can be targeted to a particular tissue or organ, such as bone marrow.

A further consideration in this aspect is directed to the timing of injection of the mesenchymal stem cells into the patient relative to the administration of ECM. In one embodiment, the mesenchymal stem cells are injected simultaneously with the ECM. The ECM and mesenchymal stem cells, for example, can be mixed ex vivo prior to being administered to the site of desired treatment. In another embodiment, the mesenchymal stem cells are administered before or after the administration of the ECM.

The ECM and mesenchymal stem cells isolated from the donor may be delivered in crude form, i.e. they may comprise other biological materials. The biological materials include, but are not limited to, vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor of matrix metalloproteinase (TIMP), interferon, interleukins, cytokines, integrin, collagen (all types), elastin, fibrillins, fibronectin, laminin, glycosaminoglycans, vitronectin, proteoglycans, transferrin, cytotactin, cell binding domains, tenascin, and lymphokines, activated platelets (which release factors that initiate the healing process), fibrin (a key component of the provisional matrix present in newly formed and healing wounds), or the like.

The ECM and mesenchymal stem cell composition may also include small molecules such as statins or hedgehog agonists, which facilitate stem cell proliferation and function. In one embodiment, these small molecules are formulated to facilitate their controlled, local release into the bone marrow, e.g., drug-loaded microspheres, matrix-bound drug, coated microspheres or other implants. The ECM composition may also contain therapeutic proteins that can be control-released locally to the bone marrow area.

6.3 Imaging In Vivo

In some embodiments, the methods are subjected to in vivo imaging, prior to the administration of ECM or ECM in combination with mesenchymal stem cells, to locate or identify sites where dysfunctional tissue or organ is present. Echo location or another imaging technique can be used to facilitate targeting. The therapeutic dose of ECM or ECM in combination with mesenchymal stem cells is then injected into the marrow cavity. magnetic resonance imaging (MRI) or single-photon emission computed tomography (SPECT) imaging can facilitate identification of the dysfunctional segments of the marrow. For example, SPECT imaging can identify areas without metabolically active cells—which would correlate to areas of dysfunction.

In these embodiments, a detectably-labeled moiety, e.g., an antibody, which is specific for an antigen in the bone marrow is administered to an individual (e.g., by injection), and labeled cells are located using standard imaging techniques, including, but not limited to MRI, magnetic resonance imaging (MRI), computer assisted tomography (CAT), magnetic resonance spectroscopy (MRS), positron emission tomography (PET), SPECT, bioluminescence image (BLI) or equivalent, and the like.

For diagnostic in vivo imaging, the type of detection instrument available is a major factor in selecting a given radionuclide. The radionuclide chosen must have a type of decay that is detectable by a given type of instrument. In general, any conventional method for visualizing diagnostic imaging can be utilized in accordance with this invention. Another important factor in selecting a radionuclide for in vivo diagnosis is that its half-life be long enough that it is still detectable at the time of maximum uptake by the target tissue, but short enough that deleterious radiation of the host is minimized.

The detectably labeled specific antibody is used in conjunction with imaging techniques, in order to analyze the expression of the target. In one embodiment, the imaging method is one of PET or SPECT, which are imaging techniques in which a radionuclide is synthetically or locally administered to a patient. The subsequent uptake of the radiotracer is measured over time and used to obtain information about the targeted tissue. Because of the high-energy (y-ray) emissions of the specific isotopes employed and the sensitivity and sophistication of the instruments used to detect them, the two-dimensional distribution of radioactivity may be inferred from outside of the body.

6.4 Decellularized Extracellular Matrix of Conditioned Body Tissue

6.4.1 Source of Body Tissue

Suitable animal body tissue from which the decellularized extracellular matrix material of the present invention is produced includes body tissues originally from syngeneic, allogeneic or xenogenic sources. The body tissue may be obtained from various animal sources. These animals include, but are not limited to, non-primate (e.g., cows, pigs, horses, chickens, cats, dogs, rats, etc.) and primate (e.g., monkeys and humans). The body tissue may be obtained at approved slaughterhouses from animals fit for human consumption or from herds of domesticated animals maintained for the purpose of providing tissues or organs. In one embodiment, the body tissue is handled in a sterile manner, and any further dissection of the body tissue is carried out under aseptic conditions. In one embodiments, the source of the body tissue is non-human. When the implants are obtained from a human, the donor may be the recipient, the donor may be genetically related to the recipient, or the donor may be unrelated to the recipient. In specific embodiments, the donor is tested for compatibility with the recipient.

Progenitor cells (e.g., endothelial progenitor cells), stem cells (e.g., mesenchymal, hematopoietic, neuronal), stromal cells, parenchymal cells, undifferentiated cells, embryonic cells, fibroblasts, macrophage, and satellite cells are particularly useful for conditioning using the methods of the present invention. In some embodiments, body organs that are useful in the present invention include, but are not limited to, brain, heart, lung, liver, pancreas, stomach, large or small intestine, kidney, bladder, uterus, bone marrow, etc.

The body tissue suitable for the present invention can be grouped into four general categories: (1) epithelial tissue, (2) connective tissue, (3) muscle tissue, and (4) nerve tissue. Epithelial tissue covers or lines all body surfaces inside or outside the body. Examples of epithelial tissue include, but are not limited to, the skin, epithelium, dermis, and the mucosa and serosa that line the body cavity and internal organs, such as the heart, lung, liver, kidney, intestines, bladder, uterine, etc. Connective tissue is the most abundant and widely distributed of all tissues. Examples of connective tissue include, but are not limited to, vascular tissue (e.g., arteries, veins, capillaries), blood (e.g., red blood cells, platelets, white blood cells), lymph, fat, fibers, cartilage, ligaments, tendon, bone, teeth, omentum, submucosa, peritoneum, mesentery, meniscus, conjunctiva, dura mater, umbilical cord, etc. Muscle tissue accounts for nearly one-third of the total body weight and consists of three distinct subtypes: striated (skeletal) muscle, smooth (visceral) muscle, and cardiac muscle. Examples of muscle tissue include, but are not limited to, myocardium (heart muscle), skeletal, intestinal wall, etc. The fourth primary type of tissue is nerve tissue. Nerve tissue is found in the brain, spinal cord, and accompanying nerve. Nerve tissue is composed of specialized cells called neurons (nerve cells) and neuroglial or glial cells.

6.4.2 Conditioning Of Body Tissue

The present invention provides methods for conditioning body tissue using one or more biological, chemical, pharmaceutical, physiological and/or mechanical manipulation. Specifically, conditioning is used to make the body tissue either over-express or underexpress a biological material of interest as compared to the amount of such biological material that the body tissue would express absent conditioning, or to express a protein or biological material otherwise not present in the tissue. In certain embodiments, the conditioning modifies the production of biological materials that enhance the effectiveness or temporal sequence of repairing, regenerating or strengthening defective, diseased, damaged or ischemic tissues or organs in a subject. In certain other embodiments, the conditioning modifies the production of biological materials that increase the metabolic synthesis of and/or phenotypic expression in endogenous cell populations. The anti-adhesion, bioadhesive, bioresorptive, antithrombogenic, and other physical properties of the body tissue can also be varied as needed by the conditioning process.

In one embodiment, the conditioning modifies the body tissue's production of extracellular matrix proteins, growth factors, angiogenesis factors, cytokines, morphogens (a biologically active material that is capable of inducing the developmental cascade of cellular and molecular events that culminate in the formation of new, organ-specific tissue), etc., and/or micro-architecture of extracellular matrix components. Examples of the biological material of interest to the present invention include, but are not limited to, vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GMCSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-I), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), tenascin, Serca2, phospholamban repressors, beta blockers, and ACE inhibitors.

More than one conditioning process may be performed, sequentially or simultaneously. The conditioning of the body tissue can be conducted in vivo, in situ or in vitro. Conditioning the body tissue while it is still in the donor animal has the advantage of retaining the complexity afforded by in vivo remodelling.

Alternatively, after the body tissue is harvested, the biologically active material composition and histoarchitectural property of the body tissue may be modified without in vivo manipulation. When conditioning is performed after the body tissue is isolated or harvested from the donor animal, i.e., in vitro, the body tissue is cultured for a period of time, for example, in a bioreactor. The advantage of in vitro conditioning is that the process is easily monitored and that changes to the biologically active material composition and histoarchitectural property of the body tissue is easily assessed.

Regardless of whether the body tissue is conditioned in vivo or in vitro, or before or after the body tissue is harvested, the conditioned body tissue should be allowed a selected period of time to produce the desired biological material in an amount different than the amount that is produced by an unconditioned body tissue. In one embodiment, the conditioned body tissue produces at least 5%, at least 10%, at least 25%, at least 50%, at least 100%, at least two times, at least five times, or at least ten times more or less biological material than a body tissue absent conditioning.

In another embodiment, the body tissue is conditioned to express a protein or biological material otherwise not present in the tissue.

In certain embodiments, the conditioned body tissue can be further processed before or after decellularization. In a specific embodiment, a therapeutic agent may be delivered to the body tissue before or after conditioning. In one embodiment, the therapeutic agent is useful for treating a disease or condition that requires tissue or organ repair, restoration and/or strengthening.

6.4.2.1 Biological Conditioning

The body tissue of a donor animal can be biologically conditioned by genetic engineering to effect a desired change in composition or amount of biologically active material in the body tissue. For instance, the body tissue may be transfected with a nucleic acid that encodes a biological material of interest (see International Publication No. WO98/28406). The body tissue of a donor animal can also be biologically conditioned using a number of in vitro culture conditions to effect changes to the histoarchitecture of the body tissue and/or composition of biologically active materials in the body tissue. In some embodiments, the in vitro biological conditioning includes the use of a bioreactor. In a specific embodiment, the conditioned body tissue is continuously cultured in the bioreactor while toxic metabolic byproducts are removed.

In general, cells in the body tissue of an animal can be transfected in vivo or in vitro with genetic material using any appropriate means such as direct injection of viral vectors, as discussed further in detail below, delivery into the local blood supply (see International Publication Nos. WO 98/58542 and WO 99/55379, each of which is incorporated herein by reference in its entirety), the use of delivery vectors (e.g., liposome) or chemical transfectants, and physico-mechanical methods such as electroporation and direct diffusion of nucleic acid. The transfected body tissue is subsequently cultured for a period of time during which the composition or amount of at least one biological material in the body tissue is changed.

For general reviews of the methods of gene transfer, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217, each of which is incorporated herein by reference in its entirety. Delivery of the nucleic acid into a donor body tissue may be either in vivo, in which case the donor body tissue is exposed to the nucleic acid or nucleic acid-carrying vector or delivery complex before being harvested from the donor animal; or in vitro, in which case, the donor body tissue may first be harvested from the donor animal and then transformed with the nucleic acid in vitro. These two approaches are known, respectively, as in vivo or in vitro gene transfer.

In one embodiment, the nucleic acid is directly administered in vivo, where it is expressed to produce a biologically active material. This can be accomplished by any of numerous methods known in the art, e.g., by constructing it as part of an appropriate nucleic acid expression vector and administering it so that it becomes intracellular, by infection using a defective or attenuated retroviral or other viral vector (see infra. and U.S. Pat. No. 4,980,286), by direct injection of naked DNA, by use of microparticle bombardment (e.g., a gene gun; Biolistic, Dupont), by encapsulation in biopolymers (poly-β-1-4-N-acetylglucosamine polysaccharide; see U.S. Pat. No. 5,635,493), by administering it in linkage to a peptide or ligand which is known to enter the nucleus, by receptor-mediated endocytosis (see, e.g., Wu and Wu, 1987, J. Biol. Chem. 262:4429-4432), or by coating with lipids.

Viral vectors include adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP 10 17 (SUPRATEK), lipids or lipoplexes, nanoparticles and microparticles with and without targeting sequences such as the protein transduction domain (PTD).

Adenoviruses, in particular, are especially attractive vehicles for delivering genes to respiratory epithelia where they cause a mild disease. Other targets for adenovirus-based delivery systems are liver, the central nervous system, endothelial cells, and muscle. The use of adenoviruses has the advantage of being capable of infecting non-dividing cells. Kozarsky and Wilson present a review of adenovirus-based gene transfer (1993, Current Opinion in Genetics and Development 3:499-503). Bout et al. demonstrate the use of adenovirus vectors to transfer genes to the respiratory epithelia of rhesus monkeys (1994, Human Gene Therapy 5:3-10). Other instances of the use of adenoviruses in gene transfer can be found in Rosenfeld et al., 1991, Science 252:431-434; Rosenfeld et al., 1992, Cell 68: 143-155; and Mastrangeli et al., 1993, J. Clin. Invest. 91:225-234. Adeno-associated virus (AAV) has also been proposed for use in gene transfer (see Walsh et al., 1993, Proc. Soc. Exp. Biol. Med. 204:289-300).

Genetically ex vivo modified cells (e.g., stem cells, fibroblasts, myoblasts, satellite cells, pericytes, cardiomyocytes, skeletal myocytes, macrophage) may be delivered to the tissue. The cells then condition the matrix.

Another way to transport the gene that encodes the biologically active material into the body tissue involves chemical or physical treatment of the cells in the body tissue to increase the potential for gene uptake and allowing the gene to be directly introduced into the nucleus or target the gene to a cell receptor. In certain embodiments, these include the use of vectors that exploit receptors on the surface of cells using liposomes, lipids, ligands for specific surface receptors, cell receptors, calcium phosphate and other chemical mediators, microinjections, electroporation, sperms, and homologous recombination. Liposomes are commercially available from Gibco BRL, for example, as LIPOFECTION® and LIPOFECTACE™, which are formed of cationic lipids such as N-[1-(2,3 dioleyloxy)-propyl]-nmnm-trimethylammonium chloride (DOTMA) and dimethyl dioctadecylammonium bromide (DDAB). Numerous methods for making liposomes are also known to those skilled in the art.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted for cell specific uptake and expression, by targeting a specific receptor (see, e.g., International Publications Nos. WO 92/06180, WO 92/22635, WO92/20316, and WO93/14188, each of which is incorporated herein by reference in its entirety). Alternatively, the nucleic acid can be introduced intracellularly and incorporated within host cell DNA for expression, by homologous recombination (Koller and Smithies, 1989, Proc. Natl. Acad. Sci. USA 8623932-8935; Zijlstra et al., 1989, Nature 342:435-438).

The invention also relates to a method for biologically conditioning body tissue by inoculating the body tissue with a solution having microorganisms, where the microorganisms are selected to produce chemicals that process the tissue. The body tissue is incubated with the inoculated microorganisms under conditions that are effective for processing the body tissue by the chemicals produced by the microorganisms. The body tissue may be subsequently treated to substantially remove or inactivate the microorganisms (see U.S. Pat. No. 6,121,041).

In other embodiments, the tissue or organ may be transformed with one or more different recombinant nucleic acid molecules, so that the cells within the tissue or organ may express at least one recombinant protein. In another embodiment, a single cell in the tissue or organ may be transfected with a single recombinant nucleic acid molecule that expresses at least one protein, which can be under the control of the same transcription control sequences or under the control of different transcription control sequences. Methods commonly known in the art of recombinant DNA technology which may be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.

In some embodiments, the invention creates in the tissue or organ localized depots for a biologically active material. The tissue or organ serves to concentrate the binding of biologically active material such as drugs that are introduced, for example, locally or systemically. This is accomplished by upregulating the production of anionic/cationic species, specific antibody recognition sequences, cell receptors, etc., in the tissue or organ. For example, the conditioned body tissue which comprises cells with a highly positively charged matrix would enhance the localization of nucleic acid at this site. This would sustain nucleic acid delivery, improve transfection and reduce degradation of the nucleic acid. In a specific embodiment, the depot would provide localization for biologically active material for the treatment of ischemia. In another specific embodiment, the depot provide localization for biologically active material listed supra and a-adrenergic blockers, P-adrenergic blockers, a-adrenergic agonists, a-1 adrenergic antagonists, AMP kinase activators, angiotensin converting enzyme (ACE) inhibitors, angiotensin I1 receptor antagonists, antiarrhythmic agents, anticoagulation agents, antiplatelet aggregation agents, antidiabetic agents, antioxidants, anti-inflammatory agents, beta blockers, bile acid sequestrants, calcium channel blockers, calcium antagonists, CETP inhibitors, cholesterol reducing agentsllipid regulators, drugs that block arachidonic acid conversion, duretics, estrogen replacement agents, inotrophic agents, fatty acid analogs, fatty acid synthesis inhibitors, fibrates, histidine, nicotine acid derivatives, nitrates, peroxisome proliferator activator receptor agonists or antagonists, ranolzine, statins, thalidomide, thiazolidinediones, thrombolytic agents, vasodilators, and vassopressors.

The form and amount of nucleic acid envisioned for use depends on the type of biologically active material and the desired effect and can be readily determined by one skilled in the art. For transfection of cells without or minimized toxic effects see U.S. Pat. No. 6,284,880.

Nucleic acids that are useful as biologically active materials for gene transfer in the present invention include, e.g., DNA and RNA sequences, that have a therapeutic or prophylactic effect after being taken up by the cells of a tissue or an organ. In one embodiment, the nucleic acid comprises an expression vector that expresses a biologically active material. In another embodiment, the nucleic acid comprises a part of an expression vector that expresses a protein or a functionally active fragment, derivative or analog thereof, or a chimeric protein (see International Publication No. WO 01/90158). The nucleic acids may encode RNA interference sequences that shut down mRNA production.

In specific embodiments, the nucleic acid encodes a sequence without a leader sequence which produces an intracellular protein. In other specific embodiments, the nucleic acid encodes a sequence with a leader sequence which produces an intercellular protein. In a specific embodiment, the nucleic acid encodes a biologically active material or a functionally active fragment, derivative or analog thereof.

In one embodiment, the nucleic acid useful in the invention encodes for polypeptides. A polypeptide is understood to be any translation product of a polynucleotide regardless of size, and whether modified or not. The polypeptide may be modified by, e.g., glycosylation, acetylation, formylation, pegylation, phosphorylation, amidation, derivatization by known protecting/blocking groups, proteolytic cleavage, linkage to a cellular ligand or other protein, etc. In specific embodiments, one or more amino acid residues in the amino acid sequence of the polypeptide, such as non-conserved amino acid residues, may include insertion, deletion and/or substitution with a different amino acid residue. These polypeptides may include, for example, those polypeptides that are biologically active in the body tissue of the donor and/or recipient animal.

The polypeptides, proteins, or functionally active fragments, derivatives, and analogs thereof, that are encoded by nucleic acids used in gene transfer include without limitation, structural proteins, growth factors and cytokines which promotes or enhances repair, regeneration or strengthening of defective, diseased, damaged or ischemic cells, tissues or organs.

In some embodiments, genes that are useful for the present invention encode proteins such as vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor of matrix metalloproteinase (TIMP), cytokines, interleukins, lymphokines, interferon, integrin, collagen (all types), elastin, fibrillins, fibronectin, heparin, laminin, glycosaminoglycans, vitronectin, proteoglycans, transferrin, cytotactin, cell binding domains (e.g., RGD), and tenascin. Other genes that are useful in the present invention include those that promote angiogenesis, modulate inflammation, and increase cell adhesion, proliferation and regeneration.

In one embodiment, genes encoding for elastin may be used to increase elastic properties of the tissue being implanted. The amount of elastin would be tailored to ultimately result in a suitable material for stent coatings, i.e., to produce an elongation property necessary to comply with stent expansion.

Antisense and ribozyme molecules which inhibit expression of a target gene can also be used in accordance with the invention. For example, in one embodiment, antisense RNA molecules which inhibit the expression of major histocompatibility gene complexes (HLA) have been shown to be most versatile with respect to modulating immune responses. Furthermore, appropriate ribozyme molecules can be designed as described, e.g., Hascloff et al., 1988, Nature 334:585-591; Zaug et al., 1984, Science 224:674-578; and Zaug and Cech, 1986, Science 231:470-475. Still further, triple helix molecules can be utilized in reducing the level of target gene activity. These techniques are described in detail by L. G. Davis et al., eds, Basic Methods in Molecular Biology, 2^{nd} ed., Appleton & Lange, Norwalk, Conn. 1994. Using any of the foregoing techniques, the expression of MHC class I1 molecules can be knocked out in order to reduce the risk of rejection of the tissue constructs described herein.

6.4.2.2 Chemical Conditioning

The body tissues may be chemically conditioned to effect a desired change in the composition of biologically active material and/or the histoarchitecture of the body tissue. In one embodiment, the body tissue may be chemically conditioned by incubating the body tissue in vitro with an isosmotic, hypotonic and/or hypertonic solution (see, e.g., U.S. Pat. No. 5,855,620 and International Publication No. WO 96/32905). Studies have shown that changes in cellular osmolality appear to directly influence cell metabolism such as lipolysis (Bilz et al., 1999, Metabolism 48(4):472-6) or protein synthesis (Schmid, 1986, Klin Wochenschr 64(1):23-8; Yates et al., 1982, J. Biol. Chem. 257(24): 15030-4).

In other embodiments, the body tissue may be detoxified with reducing agents including, for example, inorganic sulfur-oxygen ions, such as bisulfate and thiosulfate, organic sulfates, amines, ammonia/ammonium, and surfactants. Chemical solutions may also be added to modulate the salinity, pH (acidity and alkalinity), ion concentration (e.g., potassium, calcium, magnesium, phosphorous, sodium, nitrate, etc.), blood variables, plasma volume, and oxygen level of the body tissue to facilitate a change in the composition or amount of biologically active materials. In one embodiment, the body tissue is chemically conditioned to promote protein synthesis, cell proliferation, tissue regeneration and strengthening or make the cells more susceptible to biological, physiological and/or mechanical conditioning.

6.4.2.3 Pharmaceutical Conditioning

Another aspect of the invention relates to the pharmaceutical conditioning of the body tissue by delivering a therapeutic agent to the body tissue. In one embodiment, the therapeutic agent is delivered to the body tissue before the body tissue is harvested. In another embodiment, the therapeutic agent is delivered to the body tissue after the body tissue is harvested.

Therapeutic agents include those that are effective at treating, managing or preventing a disease or condition that requires tissue or organ repair, restoration and/or strengthening. Other therapeutic agents include those that that promote angiogenesis, modulate inflammation, and increase cell adhesion, proliferation and regeneration. Examples of therapeutic agents include, but are not limited to, vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor of matrix metalloproteinase (TIMP), cytokines, interleukins, lymphokines, interferon, integrin, collagen (all types), elastin, fibrillins, fibronectin, laminin, glycosaminoglycans, vitronectin, proteoglycans, transferrin, cytotactin, cell binding domains (e.g., RGD), tenascin, anti-inflammatory drugs, a-adrenergic blockers, P-adrenergic blockers, a-adrenergic agonists, a-1 adrenergic antagonists, AMP kinase activators, angiotensin converting enzyme (ACE) inhibitors, angiotensin I1 receptor antagonists, antiarrhythmic agents, anticoagulation agents, antiplatelet aggregation agents, antidiabetic agents, antioxidants, anti-inflammatory agents, beta blockers, bile acid sequestrants, calcium channel blockers, calcium antagonists, CETP inhibitors, cholesterol reducing agents/lipid regulators, drugs that block arachidonic acid conversion, duretics, estrogen replacement agents, inotropic agents, fatty acid analogs, fatty acid synthesis inhibitors, fibrates, histidine, nicotine acid derivatives, nitrates, peroxisome proliferator activator receptor agonists or antagonists, ranolzine, statins, thalidomide, thiazolidinediones, thrombolytic agents, vasodilators, vasopressors, vitamins, antioxidants, herbal extracts, metals, etc.

The body tissue may be conditioned pharmaceutically either when the donor subject is undergoing or has already undergone a medication or treatment, wherein as a result of the medication or treatment, the production of biological materials in the body tissue is effected.

6.4.2.4 Physiological Conditioning

The body tissue may be physiologically conditioned to effect a process or function of the body tissue. In particular, the body tissue may be physiologically conditioned to increase or decrease the level and/or rate of production of a biologically active material in the body tissue by subjecting the body tissue to temperature changes that affect chemical and protein synthesis in the cells (see e.g., Tibbett et al., 2002, Mycorrhiza 12(5):249-55).

In one embodiment, the body tissue is physiologically conditioned by cryopreservation and subsequent thawing of the body tissue as described in U.S. Pat. No. 6,291,240, which is incorporated by reference herein in its entirety. Specifically, cryopreservation and subsequent thawing (“cryopreservation/thaw cycle”) induced the cells of the body tissue to produce useful regulatory proteins, such as, growth factors, cytokines, and stress proteins (e.g., GRP78 and HSP90). Stress proteins are known to stabilize cellular structures and render the cells resistant to adverse conditions.

In a specific embodiment, the tissue and organs may be cryopreserved or frozen to below −150° C. to −180° C., in one embodiment, to below −50° C., and in another embodiment, to below −65° C. to −70° C. In another specific embodiment, the body tissue may be cryopreserved by adding glycosaminoglycan or other extracellular matrix proteins and using freezing schedule designed to maximize retention of tissue cell viability and biomechanical properties during and after the freezing process, and following a thawing schedule which maximizes cell viability. The cryopreserving agent comprises a cell-penetrating organic solute, such as dimethylsulfoxide, and a glycosaminoglycan, such as chondroitin sulphate, in an amount sufficient to cryopreserve the musculoskeletal tissue such as ligaments, tendons and cartilage (see International Publication No. WO 91/06213).

In yet another embodiment, the body tissue is subjected to physiological stresses such as oxygen deprivation or nutrient deficiency. The stress imposed on the tissue or organ by the oxygen or nutrient deprivation induces the production of regulatory proteins in the tissue or organ and in turn changes the compositions of the biologically active material and physical structure of the body implant.

Alternatively, U.S. Pat. No. 5,824,080 describes the use of photodynamic therapy (PDT), a technique to produce cytotoxic free radicals, to condition arterial tissues. The collagens in the matrix may be cross-linked using photooxidative catalysis and visible light and therefore, add mechanical strength and/or resilience to the body tissue.

6.4.2.5 Mechanical Conditioning

Tissues respond to mechanical forces by remodelling the extracellular matrix. The magnitude and direction of mechanical force will determine the extent and type of remodelling. For example, increased stress on bones results in an increase in bone mass. Accordingly, artificial stressing of a tissue or organ that is to be harvested for the present invention modifies the properties and compositions of biologically active materials of the tissue or organ. The mechanical force can be repeatedly applied over a period of time until the desired amount of biological active material is obtained.

In a specific embodiment, a portion of the small intestine of a donor animal, such as a pig, may be mechanically conditioned by placing a balloon inside the portion of the small intestine. The balloon is inflated such that it stretches the intestinal wall. In one embodiment, the inflation or deflation of the balloon may occur in a cyclic fashion. In one embodiment, the inflation only occurs during certain periods of time during the day, thus allowing the animal's digestive system to function normally when the balloon is deflated.

Other methods of mechanically conditioning the body tissue includes the use of standard clips to create tension in the body tissue. In another specific embodiment, the body tissue is mechanically conditioned by the application of strain, wherein cell division is facilitated and the activity of matrix metalloproteinases (MMPs) are improved (see International Publication No. WO 02/62971). In yet another specific embodiment, the body tissue is subject to a hydrostatic and/or hydrodynamic force as described in U.S. Pat. No. 6,197,296, which is incorporated herein by reference in its entirety.

In yet another specific embodiment, the body tissue is subject to electroprocessing techniques, including electrospin, electrospray, electroaerosol, and electrosputter (see International Publication Nos. WO 02/40242 and WO 02/18441). Centrifugation, electrical stimulation, electromagnetic forces (e.g., seeding tissue and/or cells with magnetic particles), hydrostatic or hydrodynamic forces, sound waves, and ultrasound waves may also be used to manipulate the amount or composition of biologically active materials in the body tissue. In a specific embodiment, the electrical stimulation is generated with conductive wires connected to an electric potential which cause changes by varying the electric field or by causing mechanical forces (e.g., muscle contraction). In another specific embodiment, the electromagnetic forces and/or strains are generated by applying an electromagnetic field. In another specific embodiment, the hydrostatic or hydrodynamic forces are generated by first inserting a catheter or cannula into the tissue or organ; then forcing saline or another biologically inert fluid into the tissue and subsequently removing the same from the tissue such that the forces from the pressurized fluid conditions the tissue. In yet another specific embodiment, the sound wave and ultrasound waves are produced by commercially available spealers or transducers.

This invention also provides an in vitro method for mechanically conditioning tissue in an oriented manner (see U.S. Pat. Nos. 5,765,350, 5,700,688 and 5,521,087). For example, connective body tissues may be aligned along a defined axis to produce an oriented tissue-equivalent having increased mechanical strength in the direction of the axis. The tensile strength of collagen in a body tissue can also be improved by cross-linking or plasticizing collagen thread or thread construct with a plasticizing agent, imparting a tensile load to the collagen thread or construct to strain the collagen thread, and then allowing the strain in the thread to decrease by stress-relaxation or by creep (see U.S. Pat. No. 5,718,012 and International Publication No. WO 97/45071). The amount of biological material may be measured before, during and/or after the conditioning.

Biological, chemical, or pharmaceutical conditioning may be enhanced by use of ultrasound or iontophoresis during delivery to the tissue to be conditioned.

6.4.3 Culturing The Conditioned Body Tissue

The conditioned body tissue may be cultured over a period of time to allow changes in the biochemical composition and histoarchitecture to occur. In one embodiment, the conditioned body tissue is allowed a period of time to produce a biological material in an amount that is different than the amount that would be produced by an unconditioned body tissue.

The period of time in culture varies depending on the type of conditioning and also the extent of change desired. In specific embodiments, the conditioned body tissue may be cultured for at least 10 minutes, at least 30 minutes, at least 1 hour, at least 2 hours, at least 3 hours, at least 4 hours, at least 6 hours, at least 8 hours, at least 12 hours, at least 24 hours, at least 2 days, at least 4 days, at least 6 days, at least 8 days, at least 10 days, at least 1 week, at least 2 weeks, at least 3 weeks, at least 1 month, at least 2 months, or at least 3 months.

For conditioning process that are carried out in vitro, the body tissue may be grown in multicavity bag or bioreactors which provide low shear to the tissue. The bioreactor designs useful for the present invention are disclosed in, e.g., U.S. Pat. Nos. 4,988,623; 5,026,650; 5,153,131; and 5,928,945. In one embodiment, a horizontal rotating wall vessel (RWV) bioreactor is used. The RWV bioreactor is described in U.S. Pat. No. 5,026,650 and is incorporated by reference herein. In some embodiments, culture medium such as supplemented Dulbeccos modified Eagle medium (DMEM) (e.g., Life Technologies, Grand Island, N.Y.) may be used.

6.4.4 Assays For Monitoring The Effects of Conditioning

Changes in the amount of biologically active material subsequent to various conditioning methods may be assayed using methods known in the art. For example, mRNA levels for any factors may be determined using standard techniques in the art such as the quantitative reverse transcript TaqManB polymerase chain reaction (QRTPCR) (see, e.g., Holland et al., 1991, Proc. Natl. Acad. Sci. USA 88:7276-7289 and Lee et al., 1993, Nucl. Acids Res. 21:3761-3766). Protein levels can also be determined by techniques such as Western blots, standard ELISA assays, and biological activity assays such as the chick chorioallantoic membrane (CAM) assay.

6.4.5 Decellularized Extracellular Matrix

Decellularization generally refers to the removal of all cells, cellular components, and other non-extracellular matrix components (e.g., serum, fat) while leaving intact an extracellular matrix (ECM) component. It is believed that the process of decellularization can reduce or eliminate immune response associated with the cells as well as the cellular components. Acellular vascular tissues have been suggested to be ideal natural biomaterials for tissue repair and engineering (Schmidt and Baier, 2000, Biomaterials 21:2215-31).

Several means of reducing the viability of native cells in tissues and organs are known, including physical, chemical, and biological methods (see, e.g. Kaushal et al., 2001, Nature Medicine 7(9):1035; Schmidt et al., supra; and U.S. Pat. No. 5,192,312, which are incorporated herein by reference). Such methods may be employed in accordance with the process described herein. However, in some embodiments, the decellularization technique employed should not result in gross disruption of the anatomy of the body tissue or substantially alter its biomechanical properties or histoarchitecture. Similarly, the treatment of the body tissue to produce a decellularized extracellular matrix should also not leave a cytotoxic environment that inhibits subsequent repopulation of the extracellular matrix with cells from a recipient after implantation of the decellularized extracellular matrix. Decellularization by physical, chemical and/or biological treatments are optimized to preserve as much as possible the biological material of interest and more importantly, the microstructure of the extracellular matrix.

Extracellular matrix may be isolated from the conditioned body tissue using a physical technique, including but not limited to centrifugation, rinsing, agitation, freeze-thaw, sedimentation, dialysis, electrical stimulation, electromagnetic forces, hydrostatic or hydrodynamic forces, blasting with sound waves, and ultrasonication. For example, the conditioned body tissue may be minced to disrupt the cell membrane and disorganize cellular components. The minced body tissue may then be centrifuged with a liquid preparation, such as Histopaque, water or saline, which separates components of different densities. In some embodiments, the speed for centrifugation ranges from 100 to 10,000 g, particularly from 2,500 to 7,500 g, for between 5 to 20 minutes. The components in the resulting suspension may then be separated using filters of specific pore size. In one embodiment, the filter is of a pore size, such as 70 to 250 pm, that allows the extracellular matrix to pass through. In another embodiment, the filter is of a pore size, such as 20 to 100 pm, that retains the extracellular matrix and larger components. Filtration is carried out in one step or a series of steps.

It has been reported that modification of the magnitude of the membrane dipole potential using compounds such as cholesterol, phloretin, and 6-ketocholestanol may also influence binding capacity and disrupts membrane domains. (Asawakarn T. et al., 2001, J. Biol. Chem. 276:38457-63). Accordingly, the present invention further relates to methods for decellularizing conditioned body tissue by agitating cellular membrane potential using electrical (e.g., voltage) means.

In another specific embodiment, the formation of intracellular ice is used to decellularize the conditioned body tissue. For example, vapor phase freezing (slow rate of temperature decline) of the body tissue reduces the cellularity of the body tissue as compared to liquid phase freezing (rapid). However, slow freezing processes, in the absence of cryoprotectant, may result in tissue disruption such as cracking. Colloid-forming materials may be added during freeze-thaw cycles to alter ice formation patterns in the body tissue. Polyvinylpyrrolidone (10% w/v) and dialyzed hydroxyethyl starch (10% w/v) may be added to standard cryopreservation solutions (DMEM, 10% DMSO, 10% fetal bovine serum) to reduce extracellular ice formation while permitting formation of intracellular ice. This allows a measure of decellularization while affording the collagenase tissue matrix some protection from ice damage.

Alternatively, the conditioned body tissue may be decellularized using a chemical technique. In one embodiment, the conditioned body tissue is treated with a solution effective to lyse native cells. In one embodiment, the solution may be an aqueous hypotonic or low ionic strength solution formulated to effectively lyse the native tissue cells. Such an aqueous hypotonic solution may be de-ionized water or an aqueous hypotonic buffer. In one embodiment, the aqueous hypotonic buffer may contain additives that provide suboptimal conditions for the activity of selected proteases, e.g., collagenase, which may be released as a result of cellular lysis. Additives such as metal ion chelators, e.g., 1,10-phenanthroline and ethylenediaminetetraacetic acid (EDTA), create an environment unfavorable to many proteolytic enzymes.

In another embodiment, the conditioned body tissue is treated with a hypotonic lysis solution with protease inhibitors. General inhibitor solutions manufactured by Sigma and Genotech are particularly useful. Specifically, 4-(2-aminoethyl)-benzene-sulfonyl fluoride, E-64, bestatin, leopeptin, aprotin, PMSF, Na EDTA, TIMPs, pepstatin A, phosphoramidon, and 1,10-phenanthroline are non-limiting examples of useful protease inhibitors. The hypotonic lysis solution may have include a buffered solution of water, pH 5.5 to 8, such as pH 7 to 8. In some embodiments, the hypotonic lysis solution is free from calcium and zinc ions. Additionally, control of the temperature and time parameters during the treatment of the body tissue with the hypotonic lysis solution, may also be employed to limit the activity of proteases.

In certain embodiments, the body tissue is treated with a detergent. In one embodiment, the body tissue is treated with an anionic detergent, such as sodium dodecyl sulfate in buffer. In another embodiment, the body tissue is treated with a non-ionic detergent, such as Triton X-100 or 1% octyl phenoxyl polyethoxyethanol, to solubilize cell membranes and fat. In one embodiment, the body tissue is treated with a combination of different classes of detergents, for example, a nonionic detergent, Triton X-100, and an anionic detergent, sodium dodecyl sulfate, to disrupt cell membranes and aid in the removal of cellular debris from tissue.

Steps should be taken to eliminate any residual detergent levels in the extracellular matrix, so as to avoid interference with the latter's ability to repair, regenerate or strengthen defective, diseased, damaged or ischemic tissues or organs. Selection of detergent type and concentration will be based partly on its preservation of the structure, composition, and biological activity of the extracellular matrix.

In other embodiments, extracellular matrix may be isolated from the conditioned body tissue using a biological technique. Various enzymes may be used to eliminate viable native cells from the body tissue. In one embodiment, the enzyme treatment limits the generation of new immunological sites. For instance, extended exposure of the body tissue to proteases such as trypsin result in cell death. However, because at least a portion of the type I collagen molecule is sensitive to a variety of proteases, including trypsin, this may not be the approach of choice for collagenous grafts intended for implant in high mechanical stress locations.

In one embodiment, the body tissue is treated with nucleases to remove DNA and RNA. Nucleases are effective to inhibit cellular metabolism, protein production, and cell division without degrading the underlying collagen matrix. Nucleases that can be used for digestion of native cell DNA and RNA include both exonucleases and endonucleases. A wide variety of which are suitable for use in this step of the process and are commercially available. For example, exonucleases that effectively inhibit cellular activity include DNase I and RNase A (SIGMA Chemical Company, St. Louis, Mo.) and endonucleases that effectively inhibit cellular activity include EcoR I (SIGMA Chemical Company, St. Louis, Mo.) and Hind III (SIGMA Chemical Company, St. Louis, Mo.). It is preferable that the selected nucleases are applied in a physiological buffer solution which contains ions, such as magnesium and calcium salts, which are optimal for the activity of the nuclease. It is also preferred that the ionic concentration of the buffered solution, the treatment temperature, and the length of treatment are selected to assure the desired level of effective nuclease activity. In one embodiment, the buffer is hypotonic to promote access of the nucleases to the cell interiors.

Other enzymatic digestion may be suitable for use herein, for example, enzymes that disrupt the function of native cells in a transplant tissue may be used. For example, phospholipase, particularly phospholipases A or C, in a buffered solution, may be used to inhibit cellular function by disrupting cellular membranes of endogenous cells. In one embodiment, the enzyme employed should not have a detrimental effect on the extracellular matrix protein. The enzymes suitable for use may also be selected with respect to inhibition of cellular integrity, and also include enzymes which may interfere with cellular protein production. The pH of the vehicle, as well as the composition of the vehicle, will also be adjusted with respect to the pH activity profile of the enzyme chosen for use. Moreover, the temperature applied during application of the enzyme to the tissue should be adjusted in order to optimize enzymatic activity.

In another embodiment, the body tissue is treated so the cells are removed using immunomagnetic bead separation techniques directed to cell surface markers (e.g., integrins, lineage markers, stem cell markers). Immunomagnetic separation (IMS) technology can isolate strains possessing specific and characteristic surface antigens (Olsvik O. et al., 1994, Clin. Microbial Rev. 7:43-54). Commercially available immunomagnetic separation processes such as Cell Release™ (Sigris Research, Brea, Calif.) was developed to address the need for a fast, general-purpose way to detach intact cells from beads after immunomagnetic separation.

Subsequent to decellularization protocols, the resultant extracellular matrix is washed at least once with suitable chemical solutions, such as saline, protease, enzymes, detergents, alcohols, acidic or basic solutions, salt solutions, etc., to assure removal of cell debris which may include cellular protein, cellular lipids, and cellular nucleic acid, as well as any extracellular debris such as lipids and proteoglycans. Removal of the cellular and extracellular debris reduces the likelihood of the extracellular matrix eliciting an adverse immune response from the recipient upon injection or implantation. For example, the tissue may be incubated in a balanced salt solution such as Hanks' Balanced Salt Solution (HBSS), such as sterile. The washing process may include incubation at a temperature of between about 2° C. and 42° C., with 4° C. to 25° C. most preferable. The transplant tissue matrix may be incubated in the balanced salt wash solution for up to 10 to 12 days, with changes in wash solution every second or third day. The composition of the balanced salt solution wash, and the conditions under which it is applied to the transplant tissue matrix may be selected to diminish or eliminate the activity of the nuclease or other enzyme utilized during the decellularization process.

Optionally, an antibacterial, an antifungal or a sterilant or a combination thereof, may be included in the balanced salt wash solution to protect the transplant tissue matrix from contamination with environmental pathogens. In certain embodiments, the ECM is sterilized by irradiation, ultraviolet light exposure, ethanol incubation (70-100%), treatment with glutaraldehyde, peracetic acid (0.1-1% in 4% ethanol), chloroform (0.5%), or antimycotic and antibacterial substances.

The extracellular matrix prepared in accordance with the above is free of its native cells, and additionally, cellular and extra-cellular antigen components have been washed out of the extracellular matrix. In one embodiment, the extracellular matrix has been treated in a manner which limits the generation of new immunological sites in the collagen matrix. In one embodiment, the ECM is obtained as a slurry of small particles. This slurry may eventually be processed into an implant. In another embodiment, the ECM is obtained as an entire or partial structure, such as a sheet, or a tubular member, such as a small intestine.

In addition, the decellularized extracellular matrix may contain a significant portion of the original tissue mass retaining physical properties in regard to strength and elasticity and has components which are largely collagens but also comprise glycosaminoglycans and proteins closely associated with collagen such as the basement membrane complex, laminin, fibronectin, growth factors, and cytokines.

One aspect of the invention further provides the preservation of the decellularized extracellular matrix for later use. The decellularized extracellular matrix can be freeze-dried for prolonged storage. Likewise, the decellularized extracellular matrix can be air-dried by any known standard techniques. In one embodiment, the decellularized extracellular matrix can be concentrated or dehydrated and later reconstituted or rehydrated, respectively, before use. In yet another embodiment, the decellularized extracellular matrix can be used to screen pathogens such as bacteria, virus, and fungus, etc.

In yet another embodiment, the decellularized extracellular matrix is lyophilized. The lyophilized ECM may be in the form of an implant which has pores. Characteristics of the pore structure can be controlled by process parameters. In yet another embodiment, the decellularized extracellular matrix is formed as a gel. In one embodiment, the proteins are temporarily and reversibly denatured. In yet another embodiment, the decellularized extracellular matrix is precipitated or co-precipitated with other proteins or biologics.

In certain embodiments, the decellularized extracellular matrix is cryopreserved. General techniques for cryopreservation of cells are well-known in the art (see, e.g., Doyle et al., (eds), 1995, Cell & Tissue Culture: Laboratory Procedures, John Wiley & Sons, Chichester; and Ho and Wang (eds), 1991, Animal Cell Bioreactors, Butterworth-Heinemann, Boston, each of which is incorporated herein by reference). In one embodiment, the tissue or organ is thawed rapidly before use, in a water bath at 34° C. to 37° C., to avoid damage to the cells. Cryopreservation of decellularized extracellular matrix would assure a supply or inventory of substantially non-immunogenic extracellular matrices which, upon thawing, would be ready for further treatment according to the subsequent steps of this invention, or further processed as desired to provide an implant tissue product. For example, extracellular matrices may be inventoried until such time as the particular cells to be employed during repopulation are identified. This may be of particular utility when the extracellular matrix is to be repopulated with cells derived from the recipient or other cells selected for use based on their immunological compatibility with a specific recipient. The ECM may also be used in combination with cells.

6.5 Uses of the Decellularized Extracellular Matrix

The present invention further provides methods for repairing, regenerating or strengthening cells, tissues or organs. In particular, the invention relates to methods for formulating the decellularized extracellular matrix as pharmaceutical compositions, body implants, tissue regeneration scaffolds, and medical devices. Although decellularized extracellular matrix of conditioned body tissue is described in detail, it is not necessary that the decellularized extracellular matrix be of conditioned body tissue. For example, the decellularized extracellular matrix may be of body tissue that has not be conditioned as described herein. Accordingly, although some of the uses of the decellularized extracellular matrix may be described as a use for a decellularized extracellular matrix of a conditioned body tissue, decellularized extracellular matrix of a non-conditioned body tissue may alternatively be used.

In certain embodiments, the decellularized extracellular matrix of body tissue, including conditioned body tissue, may be used to treat defective, diseased, damaged or ischemic tissues or organs which include, but are not limited to, head, neck, eye, mouth, throat, esophagus, chest, bone, ligament, cartilage, tendons, lung, colon, rectum, stomach, prostate, breast, ovaries, fallopian tubes, uterus, cervix, testicles or other reproductive organs, hair follicles, skin, diaphragm, thyroid, blood, muscles, bone marrow, heart, lymph nodes, blood vessels, large intestine, small intestine, kidney, liver, pancreas, brain, spinal cord, and the central nervous system.

In particular, the decellularized extracellular matrix of body tissue, including conditioned body tissue, of the present invention may be used to treat diseases that may benefit from improved angiogenesis, cell proliferation and tissue regeneration. Such diseases or conditions include, but are not limited to, burns, ulcer, trauma, wound, bone fracture, diabetes, psoriasis, arthritis, asthma, cystitis, inflammation, infection, ischemia, restenosis, stricture, atherosclerosis, occlusion, stroke, infarct, aneurysm, abdominal aortic aneurysm, uterine fibroid, urinary incontinence, vascular disorders, hemophilia, cancer, and organ failure (e.g., heart, kidney, lung, liver, intestine, etc.). Such diseases or conditions also include, but are not limited to, diseases involving insufficiency of mature or regenerating cells in tissues, diseases involving myocardial regeneration, atherosclerosis, and the like.

In a specific embodiment, the present invention regenerates or replaces at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, at least 10%, at least 5%, or at least 1% of defective, diseased, damaged or ischemic cells from the affected tissue or organ.

The methods of the present invention is provided for an animal, including but not limited to mammals such as a non-primate (e.g., cows, pigs, horses, chickens, cats, dogs, rats, etc.), and a primate (e.g. monkey such as acynomolgous monkey and a human). In one embodiment, the subject is a human.

The present invention is useful alone or in combination with other treatment modalities. In certain embodiments, the treatment of the present invention further includes the administration of one or more immunotherapeutic agents, such as antibodies and immunomodulators, which include, but are not limited to, HERCEPTIN®, RITUXAN®, OVAREX™, PANOREX®, BEC2, IMC-C225, VITAXIN™, CAMPATH® I/H, Smart M195, LYMPHOCIDE™, Smart I D10, ONCOLYM™, rituximab, gemtuzumab, or trastuzumab. In certain other embodiments, the treatment method further comprises hormonal treatment. Hormonal therapeutic treatments comprise hormonal agonists, hormonal antagonists (e.g., flutamide, tamoxifen, leuprolide acetate (LUPRON™), LH-RH antagonists), inhibitors of hormone biosynthesis and processing, steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), and antiandrogens (e.g., cyproterone acetate).

6.5.1 Pharmaceutical Compositions

The decellularized extracellular matrix of conditioned body tissue can be formulated into pharmaceutical compositions that are suitable for administration to a subject. Such compositions comprise a prophylactically or therapeutically effective amount of the decellularized extracellular matrix as disclosed herein, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete) or, such as MF59C.1 adjuvant available from Chiron, Emeryville, Calif.), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Other examples of suitable pharmaceutical vehicles are described in “Remington: the Science and Practice of Pharmacy”, 20th ed., by Mack Publishing Co. 2000.

Generally, the ingredients of compositions of the invention are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed from an infusion bottle containing sterile pharmaceutical grade water or saline. Where the composition is administered by injection, an ampule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer the compositions of the invention, e.g., encapsulation in liposomes, microparticles, microcapsules, receptor-mediated endocytosis (see, e.g., Wu and Wu, 1987, J. Biol. Chem. 262:4429-4432), etc. Methods of administering a prophylactic or therapeutic amount of the compositions of the invention include, but are not limited to, parenteral administration (e.g., intradermal, intramuscular, intracoronary, intraperitoneal, intravenous and subcutaneous), epidural, and mucosal (e.g., intranasal, inhaled, and oral routes). The composition comprising decellularized extracellular matrix of conditioned body tissue may be administered by any convenient route, for example, by infusion or bolus injection, by absorption through epithelial or mucocutaneous linings (e.g., oral mucosa, rectal and intestinal mucosa, etc.) and may be administered together with other biologically active agents, such as paclitaxel. Administration can be systemic or local. In addition, it may be desirable to introduce the pharmaceutical composition of the invention into the central nervous system by any suitable route, including intraventricular and intrathecal injection; intraventricular injection may be facilitated by an intraventricular catheter, for example, attached to a reservoir, such as an Ommaya reservoir.

In another embodiment, the decellularized extracellular matrix of conditioned body tissue can be delivered in a controlled release or sustained release system. In one embodiment, a pump may be used to achieve controlled or sustained release (see Langer, 1990, Science 249: 1527-1533; Sefton, 1987, CRC Crit. Ref: Biomed. Eng. 14:20; Buchwald et al., 1980, Surgery 88:507; Saudek et al., 1989, N. Engl. J. Med. 321 574). Any technique known to one of skill in the art can be used to produce sustained release formulations comprising the decellularized extracellular matrix of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety. In another embodiment, the decellularized extracellular matrix is configured to be controllably released. For example, the decellularized extracellular matrix may be configured to be resorbed by the body of the patient at a predetermined rate. Accordingly, the body of the patient will receive the therapeutic benefits of the decellularized extracellular matrix at the predetermined rate.

In another embodiment, the absorption rate can be adjusted using formulas comprising fibrin, or other materials such as methylcellulose, collagen, or cholesterol.

In another embodiment, polymeric materials can be used to achieve controlled or sustained release of the decellularized extracellular matrix material (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228: 190; During et al., 1989, Ann. Neurol. 25:351; Howard et al., 1989, J. Neurosurg. 71:105); U.S. Pat. Nos. 5,679,377, 5,916,597, 5,912,015, 5,989,463 and 5,128,326; International Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glucosides) (PLGA), and polyorthoesters. In one embodiment, the polymer used in a sustained release formulation is inert, free of leachable impurities, stable during storage, sterile, and biodegradable. In yet another embodiment, a controlled or sustained release system can be placed in proximity to the target, thus requiring only a fraction of the systemic dose (see, e.g., Goodson, 1984, in Medical Applications of Controlled Release, supra, vol. 2, pp. 115-138).

The amount of the pharmaceutical composition which will be effective in the treatment of a particular disorder or condition will depend on the nature of the disorder or condition, and can be determined by standard clinical techniques. In addition, in vitro assays and animal models may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

6.5.2 Body Implant

Methods of the present invention also include methods for making and implanting a body implant comprising the decellularized extracellular matrix of body tissue, including conditioned body tissue.

The body implants of the present invention may be, without limitation: (1) vascular implants, such as carotid artery replacement, and general vein and artery replacement in the body; (2) heart valves and patches; (3) burn dressings and coverings; (4) muscle, tooth and bone implants; (5) pericardium and membranes; (6) myocardial patch; (7) urethral sling; and (8) fiber for filling aneurysms.

In one embodiment, the body implant may be a tubular member. The decellularized extracellular matrix is processed into tubular or cylindrical form. The tubular member may be used, for example, to create an esophagus, a vein, an artery, or any other tubular body member. The tubular member may be implanted or placed into a damaged area of a body of a patient. The decellularized extracellular matrix will be resorbed as native or host tissue (tissue produced by the body of the patient) forms. In some cases, the host tissue is functional, vascularized, and morphologically similar to the normal tissue.

In one embodiment of the tubular member, an elastomeric biocompatible polymer is applied to the tubular member. For example, the elastomeric biocompatible polymer may be applied to the tubular member via a spray, glue or other adhesive, radio frequency welding, staples, or any other known method. The elastomeric biocompatible polymer gives the tubular member elasticity and strength.

The elastomeric biocompatible polymer may be a nonporous material or a porous material. For example, the polymer may be a porous material if exchange of fluids and nutrients with the tissues of the patient that surround the implanted tubular member is required. Additionally, the elastomeric biocompatible polymer may be biostable or degradable. For example, a biostable elastomeric biocompatible polymer is used to provide rigidity and elasticity to the tubular member throughout the life of the patient. Examples of the elastomeric biocompatible polymer are silicones (e.g. polysiloxanes and substituted polysiloxanes), polyurethanes, thermoplastic elastomers, ethylene vinyl acetate copolymers, polyolefin elastomers, styrene isobutylene block copolymers, and EPDM rubbers.

In one embodiment, the elastomeric biocompatible polymer is applied to only the outer surface of the tubular member. In another embodiment, the elastomeric biocompatible polymer is applied to only the inner surface of the tubular member. In a further embodiment, the elastomeric biocompatible polymer is applied to both the inner and the outer surfaces of the tubular member.

In one embodiment, the body implant can be an aneurysm treatment device. For example, the aneurysm treatment device may be an aneurysm coil, a seal, a patch, or a filler. In one embodiment, the aneurysm coil is made of the decellularized extracellular matrix. In another embodiment, the aneurysm coil is made of a conventional material and is coated (as described below) with the decellularized extracellular matrix.

The aneurysm treatment device may be placed or implanted into the body of a patient. For example, the aneurysm treatment device may be implanted into the body of a patient as disclosed in U.S. Pat. No. 5,951,599, which is hereby incorporated by reference in its entirety. Once the aneurysm treatment device is placed in the body of the patient, a thrombus will form in the aneurysm. The decellularized extracellular matrix of the aneurysm treatment device will facilitate the formation of functional tissue and will resorb. In one embodiment, the decellularized extracellular matrix is modified to improve the formation of functional tissue. For example, in one embodiment, the decellularized extracellular matrix is coated with a therapeutic agent to improve the formation of functional tissue.

In another embodiment, the body implant is a ventricular restraint such as a sac or a pouch. An example of a cardiac reinforcement or restraint device is disclosed in U.S. Pat. No. 6,544,168 B2, which is hereby incorporated by reference in its entirety. For example, a sheet of the decellularized extracellular matrix may be used to form a sac or a pouch configured to receive a human heart. The sac or pouch includes a cavity that is configured to receive the heart of a patient. In one embodiment, the sac or pouch has a single opening that communicates with the cavity. In another embodiment, the sac or pouch has more than one opening that communicates with the cavity. The sac or pouch may be inserted or implanted into a patient, for example after a myocardial infarction (heart attack), such that the cavity of the sac or pouch receives the heart of the patient. The sac or pouch will provide support to the heart of the patient. Specifically, the sac or pouch will physically prevent the ventricular wall from dilating during tissue remodelling. In one embodiment, the decellularized extracellular matrix will resorb naturally within several months of the implantation or placement into the patient. The mechanical properties of the sac or pouch may be controlled by laminating several sheets of the decellularized extracellular matrix together prior to formation of the sac or pouch. Additionally, the mechanical properties of the sac or pouch may be controlled by perforating the sheet or sheets of the decellularized extracellular matrix to form a mesh.

The modified body implants comprising decellularized extracellular matrix can be implanted in vivo at the site of tissue damage to promote repair, regeneration and/or strengthening. In addition, the materials and methods of this invention are useful to promote the in vitro culture and differentiation of cells and tissues.

6.5.3 Embolotherapy

One aspect of the invention provides for the use of the decellularized extracellular matrix to embolize lesions, tumors, or vessels. In one embodiment, particles of the decellularized extracellular matrix are implanted or injected into a body of a patient to embolize lesions, tumors, or vessels. For example, particles of the decellularized extracellular matrix may be implanted using the embolizing system disclosed in U.S. Pat. No. 5,895,411, which is hereby incorporated by reference in its entirety. As the particles of the decellularized extracellular matrix degrade or resorb, they will be replaced with native tissue, i.e., tissue produced by the body of the patient. In one embodiment, the particles of the decellularized extracellular matrix are suspended in a liquid. In another embodiment, the particles of the decellularized extracellular matrix are placed in the form of a bolus. In yet another embodiment, the decellularized extracellular matrix is mixed with a polymer and formed into spheres. In such an embodiment, the polymer may be biodegradable. Alternatively, the polymer is not biodegradable.

6.5.4 Tissue Regeneration Scaffold

One aspect of the invention provides for the incorporation of the decellularized extracellular matrix of body tissue, including conditioned body tissue, into a biocompatible material for implantation into a subject, such as human. In one embodiment, the biocompatible material is in the form of a scaffold.

The scaffold may be of natural collagen, decellularized, conditioned extracellular matrix, or synthetic polymer. In certain embodiments, the scaffold serves as a template for cell proliferation and ultimately tissue formation. In a specific embodiment, the scaffold allows the slow release of the decellularized extracellular matrix of the invention into the surrounding tissue. As the cells in the surrounding tissue begin to multiply, they fill up the scaffold and grow into three-dimensional tissue. Blood vessels then attach themselves to the newly grown tissue, the scaffold dissolves, and the newly grown tissue eventually blends in with its surrounding.

### 7. EXAMPLES

### 7.1 ECM Treatment

The section of marrow needing treatment lacks stem cells (i.e. replaced by adipocytes), contains dysfunctional cells, or has a dysfunctional microstructure (extracellular matrix, vascular network). In this embodiment, an extracellular matrix derived from bone marrow is implanted into the dysfunctional region. Growth factors or signaling molecules contained within the ECM provide signals to induce marrow repair. Some growth factors induce reconstitution of the marrow structure through a repair process (e.g. VEGF, FGF, PDGF, TGF). Others will stimulate stem cells resident in the marrow to re-populate the dysfunctional region (e.g. SDF). Additionally, the ECM provides a structure or microstructure to induce marrow repair. Components of the ECM which provide this function include: collagen type I & IV, laminin, glycosaminoglycans, proteoglycans and fibronectin. Direct contact with this microstructure and these bio-molecules will facilitate stem cell infiltration, migration and reconstitution of the treated region of the marrow.

After a sufficient amount of bone marrow ECM is injected or implanted into the deficient region of bone marrow, the region is replaced by bone marrow of normal cellularity, structure and function. The proper function of this bone marrow enables the body to respond better to disease and acute and chronic injury. Increased stem cell derived factor (SDF) in submucosa

### 7.2 ECM with Mesenchymal Stem Cells Treatment

Following removal of fatty marrow from the dysfunctional region (ultrasound-facilitated liposuction), a mixture of bone marrow ECM and mesenchymal stem cells is injected to fill this space. Specifically, the ECM and mesenchymal stem cells are mixed ex vivo and administered using a large gage needle (˜18 gage or larger). In one embodiment the mesenchymal stem cells are allogeneic and bone marrow ECM animal derived.

### 7.3 ECM PBMM to Enhance Restoration of Cellular Marrow

To test the hypothesis that porcine bone marrow matrix (PBMM) could enhance restoration of cellular marrow in the tibial marrow cavity of aged rats, experiments were conducted to measure bone marrow restoration in aged rats following tibial marrow ablation. Based on studies showing that PBMM is not immunogenic, aged male nude rats (6 months of age) were used for these experiments. Prior to performing these experiments, it was verified that 6-month old nude rats had yellow fat in their marrow cavities.

The purpose of this study was to characterize the model system. While there are numerous published studies describing the effects of marrow ablation on medullary bone formation and resorption in adult rats, no study has examined this in aged rats in general, or in aged nude rats, in particular. The study design included young rats and young adult rats, in order to have control groups with which to compare the treated animals, based on the hypothesis that healing in the PBMM-treated rats would be more like that seen in younger animals. The histological appearance of the marrow was examined as a function of time and the amount of bone within the marrow cavity was assessed via micro-CT so that a complete picture of events following treatment could be obtained.

Each experiment had an N of 6 rats per time point and per age group. All rats were housed for 5 days prior to surgery to acclimate them to the vivarium and to the light/dark cycle. Marrow was ablated in the right hind tibia and at 0, 7, 14, 21, 28, 35 and 42 days, rats were euthanized, and treatment and control tibias harvested. In addition, the handling properties of PBMM and the biological response to PBMM in a preliminary study using a high dose of PBMM was assessed. This group of 6 rats was euthanized at 42 days.

The extent of endosteal bone formation in the ablated limbs was determined by microCT and histomorphometry. Immediately after harvest, the bones were fixed in 70% ethanol for 24 hours and post-fixed in neutral buffered formalin. After scanning the tibia by micro-CT, the bones were decalcified and processed for paraffin embedding. Sagittal sections of the tibias were stained with haematoxylin and eosin (H&E). All sections were reviewed subjectively and have been morphometrically analyzed in the area of new bone and new marrow. The sections may be examined by immunohistochemistry for the presence of specific cell types, and stained for detection of lipid.

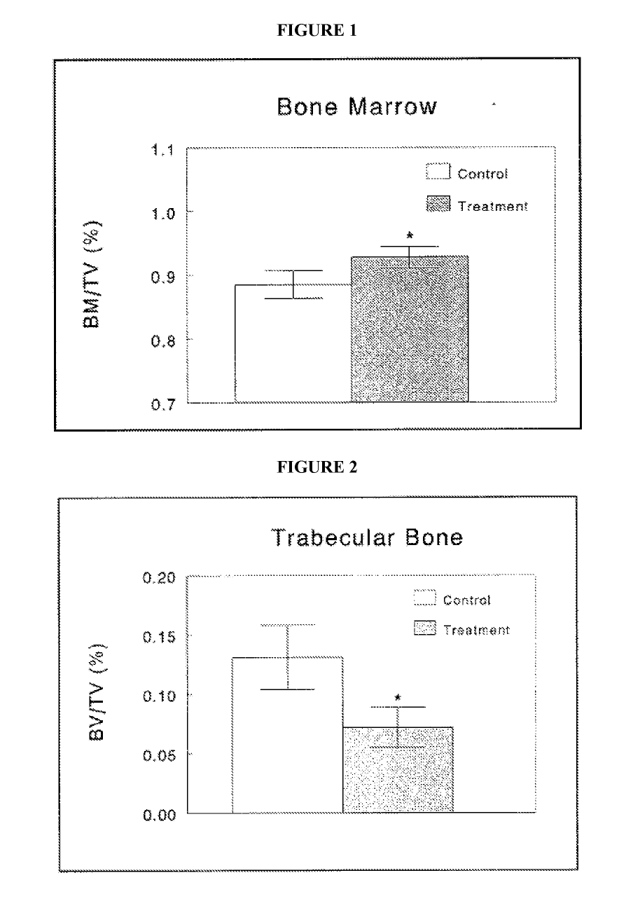

Initial review of the histologic sections of 6 month old animals euthanized 42 days after surgery demonstrated that healthy marrow was present in both control and PBMM-treated medullary canals. However, there were distinct differences between the two groups. The medullary canals of the control rat tibias contained abundant trabecular bone, whereas the medullary canals of the PBMM treated tibias exhibited much less bone trabeculae. As a result, the bone marrow compartment was increased. This observation was confirmed by micro-CT (FIGS. 1 and 2). These results strongly suggest that PBMM promotes restoration of the marrow within the medullary canal in aged nude rats. Two possible mechanisms may be involved: stimulation of bone formation and stimulation of bone remodeling. Both of these mechanisms have important implications for bone healing.

### 8. EQUIVALENTS

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

What is claimed is:

1. A method for treating a tissue or organ in a subject, comprising directly administering an effective amount of an exogenous, decellularized extracellular matrix into the site of the tissue or organ of the subject.

2. The method of claim 1, further comprising a step of examining and locating the tissue or organ prior to directly administering an effective amount of an exogenous, decellularized extracellular matrix into the site of the tissue or organ of the subject.

3. The method of claim 2, wherein the tissue or organ is examined and located by in vivo imaging.

4. The method of claim 3, wherein the tissue or organ is imaged by computer assisted tomography (CAT), magnetic resonance spectroscopy (MRS), magnetic resonance imaging (MRI), positron emission tomography (PET), single-photon emission computed tomography (SPECT), bioluminescence image (BLI) or equivalent.

5. The method of claim 1, wherein the tissue or organ is selected from the group consisting of bone marrow, spleen, liver, and thymus.

6. The method of claim 1, wherein the sites of direct administration are bone marrow cavities.

7. The method of claim 6, wherein direct administration involves injection.

8. The method of claim 6, wherein direct administration involves implantation.

9. The method of claim 1, wherein the exogenous, decellularized extracellular matrix is obtained from conditioned body tissue of an animal.

10. The method of claim 9, wherein the animal is a mammal.

11. The method of claim 10, wherein the mammal is selected from the group consisting of cows, pigs, horses, chickens, cats, dogs, rats, monkeys, and humans.

12. The method of claim 9, wherein the body tissue is selected from the group consisting of epithelial tissue, connective tissue, muscle tissue, and nerve tissue.

13. The method of claim 9, wherein the body tissue is selected from the group consisting of lymph vessels, blood vessels, heart valves, myocardium, pericardium, pericardial sac, dura mater, meniscus, omentum, mesentery, conjunctiva, umbilical cords, bone marrow, bone pieces, ligaments, tendon, tooth implants, dermis, skin, muscle, nerves, spinal cord, pancreas, gut, intestines, peritoneum, submucosa, stomach, liver, and bladder.

14. The method of claim 1, wherein the exogenous, decellularized extracellular matrix further comprises a biological material selected from the group consisting of vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor of matrix metalloproteinase (TIMP), interferon, interleukins, cytokines, integrin, collagen (all types), elastin, fibrillins, fibronectin, laminin, glycosaminoglycans, vitronectin, proteoglycans, transferrin, cytotactin, cell binding domains, tenascin, activated platelets, fibrin, and lymphokines.

15. The method of claim 1, wherein the body tissue is conditioned by a process selected from the group consisting of biological conditioning, chemical conditioning, pharmaceutical conditioning, physiological conditioning, and mechanical conditioning.

16. The method of claim 15, wherein the biological conditioning comprises transfecting the body tissue with a nucleic acid that encodes the biological material.

17. The method of claim 15, wherein the chemical conditioning comprises incubating the body tissue in a hypotonic or hypertonic solution.

18. The method of claim 15, wherein the pharmaceutical conditioning comprises delivering a therapeutic agent to the body tissue.

19. The method of claim 15, wherein the physiological conditioning comprises exposing the body tissue to heat shock or cryopreservation followed by thawing.

20. The method of claim 15, wherein the mechanical conditioning comprises applying a force to the body tissue.

21. The method of claim 20, wherein the force is selected from the group consisting of a mechanical force, centrifugal force, electrical force, electromagnetic force, hydrostatic or hydrodynamic force, sound wave, and ultrasound wave.

22. The method of claim 1, further comprising the step of removing a quantity of bone marrow from a subject to provide a space prior to injecting or implanting the exogenous, decellularized extracellular matrix.

23. The method of claim 22, wherein the bone marrow is removed by a liposuction-like technique.

24. The method of claim 22, wherein the bone marrow is removed by ultrasound energy.

25. The method of claim 22, wherein the bone marrow is removed by chemicals.

26. The method of claim 22, wherein the bone marrow is removed by radiation.

27. The method of claim 1, wherein the decellurized matrix comprises a carrier.

28. The method of claim 27, wherein the carrier is selected from the group consisting of matrix, gel, and scaffold.

29. A method for treating a tissue or organ in a subject, comprising directly administering an effective amount of exogenous, decellularized extracellular matrix in combination with mesenchymal stem cells into a bone marrow cavity.

30. The method of claim 29, further comprising a step of examining and locating the tissue or organ prior to directly administering an effective amount of exogenous, decellularized extracellular matrix in combination with mesenchymal stem cells into the bone marrow cavity.

31. The method of claim 30, wherein the bone marrow cavity is examined and located by in vivo imaging.

32. The method of claim 31, wherein the bone marrow cavity is imaged by computer assisted tomography (CAT), magnetic resonance spectroscopy (MRS), magnetic resonance imaging (MRI), positron emission tomography (PET), single-photon emission computed tomography (SPECT), bioluminescence image (BLI) or equivalent.

33. The method of claim 29, wherein the exogenous, decellularized extracellular matrix and the mesenchymal stem cells are mixed ex vivo prior to administration.

34. A method for treating and/or preventing diseases involving insufficiency of mature or regenerating cells in tissues, comprising administering to a patient in need thereof exogenous, decellularized bone marrow extracellular matrix into a bone marrow cavity.

35. A method for treating and/or preventing diseases involving myocardial regeneration, comprising administering to a patient in need thereof exogenous, decellularized bone marrow extracellular matrix into a bone marrow cavity.

36. A method of regenerating the endothelium, comprising administering to a patient in need thereof exogenous, decellularized bone marrow extracellular matrix into a bone marrow cavity.

37. A method of treating and/or preventing atherosclerosis, comprising administering to a patient in need thereof exogenous, decellularized extracellular matrix into a bone marrow cavity.

38. A method of treating cancer and/or improving recovery following chemotherapy or radiation therapy, comprising administering to a patient in need thereof, exogenous, decellularized extracellular matrix into a bone marrow cavity.
